SEQUENCE LISTING (13 sequences)

SEQ ID NO: 1 - protein, 17 aa, artificial
  calmodulin-binding peptide
KRPMNAFIVWSRDQRRK

SEQ ID NO: 2 - protein, 17 aa, artificial
  calmodulin-binding peptide
KRPMNAFMVWSRGQRRK

SEQ ID NO: 3 - protein, 17 aa, artificial
  calmodulin-binding peptide
KRPMNAFMVWSRAQRRK

SEQ ID NO: 4 - protein, 17 aa, artificial
  calmodulin-binding peptide
KRPMNAFMVWSQIERRK

SEQ ID NO: 5 - protein, 17 aa, artificial
  calmodulin-binding peptide
KRPMNAFMVWSKIERRK

SEQ ID NO: 6 - protein, 17 aa, artificial
  calmodulin-binding peptide
KRPMNAFMVWSQHERRK

SEQ ID NO: 7 - protein, 17 aa, artificial
  calmodulin-binding peptide
KRPMNAFMVWAKDERRK

SEQ ID NO: 8 - protein, 17 aa, artificial
  calmodulin-binding peptide
KRPMNAFMVWAQAARRK

SEQ ID NO: 9 - protein, 17 aa, artificial
  calmodulin-binding peptide
RRPMNAFMVWAKDERKR

SEQ ID NO: 10 - protein, 16 aa, artificial
  calmodulin-binding peptide
KRPMNAFMVWSSAQRR

SEQ ID NO: 11 - protein, 15 aa, artificial
  calmodulin-binding peptide
KRPMNAFMVWARIHR

SEQ ID NO: 12 - protein, 8 aa, artificial
  calmodulin-binding peptide
DYKDDDDK

SEQ ID NO: 13 - protein, 30 aa, artificial
  calmodulin-binding peptide
KETWWETWWTEWWTEWSQGPGRRRRRRRRR

(19) United States
(12) Patent Application Publication
(10) Pub. No.: US 2013/0266663 A1
(43) Pub. Date: Oct. 10, 2013

(54) SOX9 INHIBITORS

(71) Applicants: Arthur Brown, London (CA); Sandy Gian Vascotto, Thedford (CA)

(72) Inventors: Arthur Brown, London (CA); Sandy Gian Vascotto, Thedford (CA)

(21) Appl. No.: 13/695,319

(22) PCT Filed: Apr. 29, 2011
(86) PCT No.: PCT/CA11/00504

§ 371 Date: Oct. 30, 2012

Related U.S. Application Data
(60) Provisional application No. 61/329,745, filed on Apr. 30, 2010.

(51) Int. Cl. C07K 7/08; A61K 31/18; A61K 38/13; A61K 47/48; A61K 31/4745; A61K 33/24; A61K 31/69; A61K 31/5415; A61K 31/138

(52) U.S. Cl. CPC C07K 7/08; A61K 31/5415; A61K 31/18; A61K 38/13; A61K 31/138; A61K 31/4745; A61K 33/24; A61K 31/69; A61K 47/48315
USPC 424/617; 514/226.2; 514/602; 514/21.4; 514/20.5; 514/651; 514/279; 514/64; 530/300; 530/326; 514/17.7; 514/17.8; 514/17.9

(57) ABSTRACT

Methods for treating a condition associated with proteoglycan production in a mammal are provided. The methods comprise the administration of at least one of a calmodulin antagonist, a transient receptor potential (TRP) channel inhibitor and a calmodulin-binding peptide to the mammal.

### FIELD OF THE INVENTION

The present invention relates to inhibition of SOX9 to treat certain undesirable and pathological conditions, and in particular, relates to the use of known and novel compounds to inhibit SOX9.

### BACKGROUND OF THE INVENTION

Spinal cord injury (SCI) is a catastrophic event that is a major health care issue, causing lifelong disability. In the USA and Canada, more than 12,000 spinal cord injuries occur annually, and ˜275,000 people live with permanent, serious disabilities due to SCI (Univ. of Alabama Nat. SCI Stat. Cntr. and the Cdn. Paraplegic Assoc.). It has been estimated that the impact of neurotrauma is one of the single most costly occurrences in Ontario's health system. Currently, there are no effective treatments for SCI. The absence of axonal regeneration after SCI has been attributed to axon-repelling molecules in the damaged myelin and scar. Chief amongst the inhibitory molecules in the scar are chondroitin sulfate proteoglycans (CSPGs) produced by reactive astrocytes responding to the injury. However, astrocytes have also been shown to secrete ECM molecules conducive to axonal growth such as laminin and fibronectin. Thus, astrocytes produce both regeneration inhibiting and promoting molecules and successful regeneration may depend on the balance of these molecules.

While many different CSPGs are expressed after SCI (e.g. NG2, neurocan, phosphocan, brevican and versican), all rely on the same enzymes, xylosyltransferase-I and -II (XT-I, XT-II) and chondroitin 4-sulfotransferase (C4ST) to add axon-repelling chondroitin sulfate side chains to their core proteins. Chondroitin sulfate side chain synthesis is initiated by the addition of a xylose onto a serine moiety of the core protein. This function is carried out by the enzyme xylosyltransferase (XT) that exists in two isoforms encoded by two different genes XT-I and XT-II. These side chains are subsequently sulfated by chondroitin 4-sulfotransferase (C4ST). The crucial role played by these chondroitin sulfate side chains in axon repulsion is underscored by the observation that digestion of these side chains by the enzyme chondroitinase or interference with their synthesis by inhibiting XT-I increases axonal regeneration in rodent models of SCI. SOX9 is a transcription factor that up-regulates the expression of XT-I, XT-II and C4ST and down-regulates the expression of laminin and fibronectin in reactive astrocytes.

It has been determined that enzymatic digestion of the chondroitin sulfate side chains found on all CSPGs after SCI resulted in improvement in recovery and regeneration after SCI. Another group used a ribozyme directed against the mRNA encoding an enzyme necessary for CSPG production to promote sensory axon regeneration after SCI. The effect of increasing laminin in the injured rat spinal cord by intrathecal administration of laminin γ1 into the lesion was also shown to improve regeneration.

XT-I, XT-II and C4ST are expressed in similar patterns after SCI: It has been demonstrated that genes with related function may be regulated together as gene batteries after SCI. As such it has been hypothesized that, in astrocytes, genes that promote axon regeneration and genes that inhibit axon regeneration would be differentially regulated. The expression of an XT-I, XT-II and C4ST battery was assessed by real-time quantitative PCR (Q-PCR) after SCI in the rat. XT-I, XT-II and C4ST all showed similar patterns of gene expression after SCI as detected by Q-PCR. Increases in the expression of XT-I, XT-II and C4ST after SCI were accompanied by increases in the level of CSPGs when measured by slot blot analysis using protein extracts from the spinal lesion and an antibody, CS56, that recognizes an epitope common to many CSPGs. Q-PCR also demonstrated that laminin and fibronectin mRNA levels were elevated early but not late after SCI.

SOX9 modulates the expression of CSPG synthesizing enzymes and growth promoting extracellular matrix proteins. It has previously been demonstrated that SOX9 is a transcription factor that up-regulates the expression of XT-I, XT-II and C4ST and down-regulates the expression of laminin and fibronectin in reactive astrocytes. CMV-driven SOX9 expression in primary rat astrocytes resulted in significant increases in XT-I, XT-II and C4ST but not laminin or fibronectin mRNA levels, while small interfering RNA (siRNA) targeting SOX9 resulted in a 75±12% reduction in SOX9 mRNA levels and a 71±5.5% reduction in XT-I mRNA (similar reductions were observed in XT-II and C4ST expression). SOX9 knock-down did not decrease laminin or fibronectin gene expression but rather increased the expression of these genes in the untreated primary astrocyte cultures. These results demonstrated that SOX9 up-regulates XT-I, XT-II and C4ST expression while decreasing the expression of laminin and fibronectin. SOX9 is expressed in astrocytes of human disease associated with CNS scarring. To assess the potential role of SOX9 in human CNS injury and disease, SOX9 expression in cases of human hemorrhagic stroke, ischemic stroke, traumatic brain injury and SCI was surveyed and was found to be expressed in reactive astrocytes in these conditions.

Currently there is no therapy or approved strategy for promoting regeneration following CNS injury or disease. Typical experimental approaches to treating spinal cord or brain injury include: limit the immune response (i.e. cellular immunotherapies such as Proneuron—PN277), limit apoptosis and cytotoxic cascade (e.g. using Cethrin, Neotrofin), or regeneration via cell replacement (e.g. stem cell-based). The former two strategies rely on a very limited window of time in which treatment must occur, with minimization of scar production being a secondary effect. Many strategies focus almost exclusively on blocking nerve repelling molecules (NOGO, MAG) and not on increasing pro-regenerative molecules.

Accordingly, in view of the foregoing, it is clear that there is a need to develop effective therapies to treat CNS injury and disease.

### SUMMARY OF THE INVENTION

It has now been found that inhibition of SOX9 is effective to treat conditions associated with proteoglycan production or modulation and compounds useful to regulate Sox9 activity have been identified.

Thus, in one aspect, a method of treating a condition associated with proteoglycan production or modulation in a mammal is provided comprising administering to the mammal a calmodulin antagonist.

In another aspect of the invention, a method of treating a condition associated with proteoglycan production or modulation in a mammal is provided comprising administering to the mammal a compound that antagonizes calmodulin and modulates the immune response.

In another aspect of the invention, a method of treating a condition associated with proteoglycan production or modulation in a mammal is provided comprising administering to the mammal a calcium channel antagonist.

In a further aspect of the invention, a method of treating a condition associated with proteoglycan production or modulation in a mammal is provided comprising administering to the mammal a transient receptor potential (TRP) channel inhibitor.

In another aspect of the invention, a method of treating a condition associated with proteoglycan production or modulation in a mammal is provided comprising administering to the mammal a calmodulin-binding peptide.

These and other aspects of the invention are described herein by reference to the following figures.

### BRIEF DESCRIPTION OF THE FIGURES

FIG. 1 is a graphic representation of the sequence analysis of the promoter regions of human, rat, and mouse XT-I, XT-II and CX4ST illustrating positioning and other features;

FIG. 2 graphically illustrates Sox9 CSPG target gene expression (XT1, XTII, C4ST) following spinal cord injury;

FIG. 3 illustrates that Tamoxifen administration to conditional Sox9 knockout mice that are subsequently subject to spinal cord injury reduces the frequency of SOX9 expressing cells in the lesion (A) and in the spinal cord (B) and reduces frequency of GFAP positive cells (C); a correlation between the frequency of GFAP expressing cells and SOX9 expressing cells is determined (D); but does not appear to impact the frequency of astrocytes (no difference in frequency of glutamine synthetase positive cells and wildtype SOX9 knockouts (E)) as confirmed by linear regression analysis (F), and the impact of SOX9 knockout on expression of SOX9 genes at SCI (G);

FIG. 4 illustrates that Tamoxifen administration to primary astrocytes derived from conditional Sox9 knockout mice reduces the expression of SOX9 target gene expression (A), and the impact of SOX9 activity reduction upon the of scar gene expression in vitro in SOX9 knockdown (B);

FIG. 5 illustrates the results of a luciferase assay of SOX9 activity in aged astrocyte cultures treated with various concentrations of compounds designed to inhibit at least one of calcium influx, and calmodulin activity (A-E);

FIG. 6 illustrates the results of real time PCR analysis of SOX9 target gene expression and Western Blotting of samples from aged astrocyte cultures treated with various concentrations of compounds designed to inhibit at least one of calcium influx, and calmodulin activity (A, B and C);

FIG. 7 illustrates the results of real time PCR analysis of SOX9 target gene expression of samples from aged astrocyte cultures treated with cal-TAT (A/C) and TAT-cal peptide (B);

FIG. 8 illustrates the results of real time PCR analysis of SOX9 target gene expression of samples from rat spinal cord following spinal cord injury and treatment with chlorpromazine (A) and cyclosporine A treatment (B);

FIG. 9 demonstrates the trend in the behavioral improvement in mice following spinal cord injury on modulation of SOX9 at 4 weeks (A) and longer term (B), as well as measuring distance travelled to determine improvement (C);

FIG. 10 illustrates the results of histological analysis of SOX9 target gene expression of samples from rat spinal cord following spinal cord injury which show decreased CSPG expression (A), increased laminin (B) and increased neurafilament expression (C);

FIG. 11 illustrates a trend in behavioral improvement with the modulation of SOX9;

FIG. 12 illustrates the results of real time PCR analysis of SOX9 target gene expression (A) in samples from spinal cord injured mice treated with chlorporomazine by IP injection, including SOX9 (B), GFAP (C), XT-1 (D), HAPLNI (E), type 2A Collagen (F), Aggrecan (G) and Brevican (H);

FIG. 13 illustrates the results of real time PCR analysis of SOX9 target gene expression of samples from spinal cord injured mice treated with various concentrations of chlorpromazine; and

FIG. 14 illustrates a trend of behavioral improvement in CNS injured rats treated with various concentrations of chlorpromazine as shown in locomoter function (A) and grip (B) tests.

### DETAILED DESCRIPTION OF THE INVENTION

Compounds useful to regulate SOX9 activity and treat a condition associated with proteoglycan production in a mammal are provided, including calmodulin antagonists, transient receptor potential (TRP) channel inhibitors and a novel family of calmodulin-binding peptides.

SOX9 is a transcription factor required for chondrocyte differentiation and cartilage formation. In humans, SOX9 is a 56 Kda protein having 509 amino acids (NCB1 accession no. NP _{—}000337.1). SOX9 protein and nucleic acid sequences, including human and other mammalian SOX9 sequences, are well-known in the art, see for example, WO 2008/049226, the contents of which are incorporated herein by reference. Examples of SOX9 protein variant sequences include SOX9 in dog (NCB1 accession NP_{—}001002978), chimpanzee (NCB1 accession no. NP_{—}001009029.1) and mouse (NCB1 accession no. NP_{—}035578.2). For the purposes of the present invention, the term “SOX9” encompasses any functional mammalian SOX9 protein including functional variants thereof. The term “functional variant” refers to a SOX9 protein that retains the activity of a native, naturally occurring SOX9 protein, for example, regulation of a xylosyltransferase such as XT-1 or a sulfotransferase such as C4ST.

The term “proteoglycan” refers to a family of glycoproteins comprising a core protein and one or more covalently linked glycosaminoglycan chains which are formed, at least in part, by the action of a xylosyltransferase and sulfotransferase. Examples of such proteoglycans include chondroitin sulfate proteoglycans (CSPGs) with core proteins such as phosphan, NG2 and brevican; dermatan sulfate proteoglycans (DSPGs) with core proteins such as decorin; heparin sulfate proteoglycans (HSPGs) with core proteins such as syndecans, glypicans, perlecan, agrin and collagen XVII; and keratin sulfate proteoglycans (KSPGs) with core proteins such as Lumican, Keratocan, Mimecan, Fibromodulin, PRELP, Osteoadherin and Aggrecan. Xylosyltransferases for example, XT-I or XT-II catalyze the first and rate limiting step in the addition of glycosaminoglycan chains to the proteoglycan core protein by the addition of xylose.

The term “production or modulation” as it relates to proteoglycans, and conditions associated therewith, refers to the transcriptional regulation of a molecule that modifies or regulates proteoglycan activity wherein the molecule includes, but is not limited to, the core proteoglycan protein, the glycosaminoglycan chains and proteoglycan-synthesizing enzymes such as XT-I, XT-II and C4ST.

The term “conditions associated with proteoglycan production or modulation” is used herein to encompass undesirable conditions and pathologies to which proteoglycan production/modulation contributes and in which reduction of at least one proteoglycan ameliorates the condition or pathology. For example, proteoglycan production, such as production of CSPG, is known to contribute to conditions in which normal neuronal growth or neuronal plasticity, including neuronal regeneration, is blocked or otherwise impeded. Examples of such conditions include, but are not limited to, primary conditions of the nervous system that include but are not limited to, spinal cord injury, traumatic brain injury, neurodegenerative diseases, such as Friedreich's ataxia, spinocerebellar ataxia, Alzheimer's disease, Parkinson's Disease, Lou Gehrig's Disease (ALS), demyelinative diseases, such as multiple sclerosis, transverse myelitis resulting from spinal cord injury, inflammation, and diseases associated with retinal neuronal degeneration such as age-related amblyopia, maculopathies and retinopathies such as viral, toxic, diabetic and ischemic, inherited retinal degeneration such as Kjellin and Barnard-Scholz syndromes, degenerative myopia, acute retinal necrosis and age-related pathologies such as loss of cognitive function. Examples also include conditions that cause cerebrovascular injury including, but not limited to, stroke, vascular malformations, such as arteriovenous malformation (AVM), dural arteriovenous fistula (AVF), spinal hemangioma, cavernous angioma and aneurysm, ischemia resulting from occlusion of spinal blood vessels, including dissecting aortic aneurisms, emboli, arteriosclerosis and developmental disorders, such as spina bifida, meningomyolcoele, or other causes.

The utility of selected compounds to antagonize SOX9 function is readily confirmed using in vitro assay systems, for example, using cells transfected with a reporter construct activated only in the presence of SOX9, e.g. comprising one or more SOX protein binding sites functionally linked to a promoter that controls the expression of a reporter gene, e.g. luciferase. CSPG-related gene expression can also be monitored to determine the utility of a compound as a SOX9 antagonist such that a decrease in CSPG-related gene expression indicates antagonistic activity. As one of skill in the art will appreciate, suitable in vivo models may also be used to determine the utility of a potential SOX9 antagonist or inhibitor.

In one aspect of the invention, a method of treating a condition associated with proteoglycan or modulation in a mammal comprises administering to the mammal a calmodulin antagonist. Suitable calmodulin antagonists include compounds effective to inhibit, or at least reduce, SOX9 nuclear translocation. Examples of suitable calmodulin antagonists include alpha-adrenergic blockers such as phenoxybenzamine, Prazosin, Terazosin, Doxazosin, Tamsulosin and derivatives thereof such as pharmaceutically acceptable salts; phenothiazines such as chlorpromazine, calmidazolium, E6 Berbamine, CGS 9343B, trifluoperazine and fluphenazine and structurally similar cyclic polypeptides such as cyclosporine, rapamycin, and FK506, and derivatives thereof such as pharmaceutically acceptable salts; naphthalenesulfonamides such as A7, J8, W-5, W-7, W-13 and derivatives thereof such as pharmaceutically acceptable salts, e.g. HCl salts; and ACE inhibitors such as Losartan, Valsartan, Irbesartan, Candesartan and derivatives thereof such as pharmaceutically acceptable salts; and alkaloids such as Tetrandrine. As one of skill in the art will appreciate, many of such calmodulin antagonists are commercially available, or can be readily synthesized using known chemical synthetic techniques.

In another aspect of the invention, a method of treating a condition associated with proteoglycan production or modulation in a mammal comprises administering to the mammal a transient receptor potential (TRP) channel inhibitor. Suitable TRP channel inhibitors include compounds effective to inhibit, or at least reduce, calcium influx at a TRP channel, such as a TRPV channel, and thus, inhibit calmodulin capacity to transport SOX9. Examples of such inhibitors include broad spectrum TRP channel antagonists such as 2-APB and TRPV antagonists such as ruthenium red, citral, RN9893 and RN1734, and derivatives thereof such as pharmaceutically acceptable salts. As one of skill in the art will appreciate, TRP channel inhibitors are commercially available, or can be readily synthesized.

In a further aspect of the invention, a method of treating a condition associated with proteoglycan production or modulation in a mammal comprises administering to the mammal a calmodulin-binding peptide. Suitable calmodulin-binding peptides include peptides comprising an amino acid sequence sufficient to bind calmodulin.

The term “amino acid” as used herein refers to naturally occurring and synthetic amino acids in either D- or L-form. As one of skill in the art will appreciate, amino acids include: glycine; those amino acids having an aliphatic side chain such as alanine, valine, norvaline, leucine, norleucine, isoleucine and proline; those having aromatic side-chains such as phenylalanine, tyrosine and tryptophan; those having acidic side chains such as aspartic acid and glutamic acid; those having side chains which incorporate a hydroxyl group such as serine, homoserine, hydroxynorvaline, hydroxyproline and threonine; those having sulfur-containing side chains such as cysteine and methionine; those having side chains incorporating an amide group such as glutamine and asparagine; and those having basic side chains such as lysine, arginine, histidine, and ornithine.

An appropriate calmodulin-binding peptide according to the invention is represented by the following general formula:

X¹RP−spacer−RX¹X²

wherein X¹ is a positively charged amino acid such as arginine (R), lysine (K) or histidine (H); X² is a positively charged amino acid such as arginine (R), lysine (K) or histidine, or is no amino acid; and the spacer comprises from about 8-12 amino acid residues.

Examples of suitable calmodulin-binding peptides comprise a calmodulin binding site derived from a mammalian SOX protein, such as SOX1, SOX2, SOX3, SOX4, SOX5, SOX6, SOX7, SOX8, SOX9, SOX10, SOX11, SOX12, SOX13, SOX14, SOX15, SOX17, SOX18 and SOX30, and includes functionally equivalent variants of a SOX protein that retains the ability to bind calmodulin. A functionally equivalent variant SOX protein, for example, is a protein that may include one or more amino acid substitutions, additions, deletions or derivatizations while retaining the ability to bind calmodulin.

In one embodiment, the calmodulin-binding peptide is selected from the group consisting of:

KRPMNAFIVWSRDQRRK,
KRPMNAFMVWSRGQRRK,
KRPMNAFMVWSRGQRRK,
KRPMNAFMVWSRGQRRK,
KRPMNAFMVWSRGQRRK,
KRPMNAFMVWSRAQRRK,
KRPMNAFMVWSQIERRK,
KRPMNAFMVWSKIERRK,
KRPMNAFMVWSQHERRK,
KRPMNAFMVWAKDERRK,
KRPMNAFMVWAKDERRK,
KRPMNAFMVWAKDERRK,
KRPMNAFMVWAQAARRK,
KRPMNAFMVWAQAARRK,
KRPMNAFMVWAQAARRK,
RRPMNAFMVWAKDERKR,
RRPMNAFMVWAKDERKR,
RRPMNAFMVWAKDERKR,
KRPMNAFMVWSSAQRR
and
KRPMNAFMVWARIHR.

Calmodulin binding peptides in accordance with the invention can readily be made using well-established techniques for making peptides.

In synthesizing such a peptide, as one of skill in the art will appreciate, it may be advantageous to incorporate N- or C-terminal protecting groups which serve to protect the amino and carboxyl termini of the peptide from undesired biochemical attack. Useful N-terminal protecting groups include, for example, lower alkanoyl groups of the formula R—C(O)— wherein R is a linear or branched lower alkyl chain comprising from 1-5 carbon atoms. A preferred N-terminal protecting group is acetyl, CH3-C(O)—. Also useful as N-terminal protecting groups are amino acid analogues lacking the amino function. C-terminal protection may be achieved by incorporating the blocking group via the carbon atom of the carboxylic function, for example to form a ketone or an amide, or via the oxygen atom thereof to form an ester. Thus, useful carboxyl terminal protecting groups include, for example, ester-forming alkyl groups, particularly lower alkyl groups such as e.g., methyl, ethyl and propyl, as well as amide-forming amino functions such as primary amine (—NH2), as well as monoalkylamino and dialkylamino groups such as methylamino, ethylamino, dimethylamino, diethylamino, methylethylamino and the like. C-terminal protection can also be achieved by incorporating as the C-terminal amino acid a decarboxylated amino acid analogue, such as agmatine. Of course, N- and C-protecting groups of even greater structural complexity may alternatively be incorporated, if desired.

In addition, it may be desirable to modify the peptide to incorporate means to assist in the delivery of the peptide to a target site on administration. For example, the peptide may be fused to another peptide to facilitate delivery, such as the TAT sequence, and other cell penetrating peptides which belong to the family of primary amphipathic peptides, such as MPG, Pep-1 and Wr-T (KETWWETWWTEWWTEWSQGPGrrrrrrrrr (r, D-enantiomer arginine) (SEQ ID NO:13).

The present methods may utilize a selected inhibitor, e.g. a calmodulin antagonist, a transient receptor potential (TRP) channel inhibitor or a calmodulin-binding peptide, alone or in the form of a composition in which the inhibitor is combined with at least one pharmaceutically acceptable carrier or adjuvant. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with a particular type of compound, and may include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

The selected SOX9 inhibitor may also be formulated to facilitate its delivery to a target site on administration, for example, within liposomes or other formulations suitable to encapsulate the inhibitor.

In accordance with the invention, a therapeutically effective amount of a selected SOX9 inhibitor is administered to a mammal in the treatment of an undesirable condition associated with proteoglycan production or modulation. As used herein, the term “mammal” is meant to encompass, without limitation, humans, domestic animals such as dogs, cats, horses, cattle, swine, sheep, goats and the like, as well as wild animals. The term “therapeutically effective amount” is an amount of the selected SOX9 inhibitor indicated for treatment of a given condition while not exceeding an amount which may cause significant adverse effects. Suitable dosages of the selected SOX9 inhibitor will vary with many factors including the particular condition to be treated and the individual being treated. Appropriate dosages are expected to be in the range of about 1 ug-100 mg.

Administration of the SOX9 inhibitor to a mammal may be by any suitable administrable route including enterally, e.g. orally, or parenterally, e.g. intravenously, intraperitonally, intramuscularly, intrathecally and by inhalation via an appropriate carrier or matrix material.

Treatment of an undesirable condition associated with proteoglycan production/modulation using of a SOX9 inhibitor in accordance with the present invention, e.g. a calmodulin antagonist, a transient receptor potential (TRP) channel inhibitor or a calmodulin-binding peptide, may be augmented by utilizing a combination of two or more of a calmodulin antagonist, a transient receptor potential (TRP) channel inhibitor and a calmodulin-binding peptide. In addition, the present treatment methods may be used to complement other treatment approaches, including cell-based therapies or approaches that limit the immune response or cytotoxicity.

Embodiments of the invention are described by reference to the following specific examples which are not to be construed as limiting.

### EXAMPLE 1

### Astrocyte Culture System

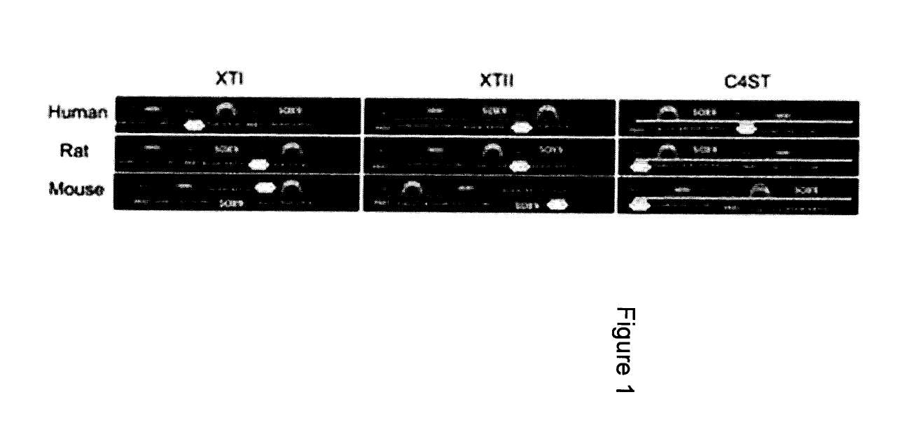

The transcription factor, SOX9, has been determined to up-regulate the transcription of XT-I, XT-II and C4ST in primary astrocyte cultures and to down-regulate the expression of the pro-regenerative extracellular matrix (ECM) molecules, laminin and fibronectin. Consistent with the hypothesis that the CSPG genes are co-regulated, Genomatix software analysis identified 5 transcription factors with putative binding sites in all 9 of their respective promoters. The relative positioning and features of these units are illustrated in FIG. 1.

To determine whether the SOX9 up-regulation of XT-I, XT-II and C4ST expression is direct or indirect, chromatin immunoprecipitation (ChIP) assays were conducted. Chromatin immunoprecipitation (ChIP) using an anti-SOX9 antibody on cells from the gonadal ridge of either female (non-SOX9 expressing) or male (SOX9-expressing) mice demonstrates that SOX9 binds to the promoter regions of C4ST and XT-l. DNA immunoprecipitated by the anti-SOX9 antibody was amplified by PCR using standard conditions and primer pairs flanking the 2 putative SOX9 binding sites in the C4ST promoter and the 3 putative SOX9 binding sites in the XT-1 promoter. Both predicted SOX9 binding sites in the C4ST promoter (at 5432 bp and 2.1 kb upstream of the transcriptional start site were amplified preferentially from the male versus female CHiPed DNA as visualized by agarose gel electrophoresis. Only one of the three predicted SOX9 binding sites in the XT-1 promoter demonstrated enrichment in the PCR-amplified male ChiPed DNA (a site 70 bp upstream of the transcriptional start site). This indicates that SOX9 directly activates the expression of XT-I, XT-II and C4ST. Genomic DNA (without immunoprecipitation) was amplified with all primer sets as a positive control for the PCR reactions.

A cell-based screen to identify inhibitors of SOX9 has been developed using primary astroctye cells. Astrocyte cultures aged in vitro have been demonstrated and generally accepted to well represent astrocytes within the mature brain. Cultures early after plating bear characteristics of immature and reactive astroctyes associated with acute damage. In this assay, primary astrocytes are transfected with a SOX9 reporter construct that consists of 4 repeats of the SOX9 binding site coupled to the mouse Col2a1 minimal promoter cloned upstream of a luciferase gene in the plasmid pGL4, as previously described in WO 2008/049226. After normalization for transfection efficiency, SOX9 activity can be monitored in transfected astrocytes by luciferase activity. Cells transfected with the SOX9 reporter construct were cultured in the presence and absence of potential inhibitors for 24 hours at which time the cells were lysed and luciferase levels measured. Compounds that reduced the levels of luciferase activity relative to control wells were considered as positive “hits”. In a secondary screen, false positives that cause a reduction in luciferase activity due to effects on cell viability, were eliminated using a cell viability assay (CellTiter-Flour—Promega). Subsequent to secondary screening, the “hits” were evaluated for their affect on SOX9 target gene expression by Q-PCR studies in primary astrocytes.

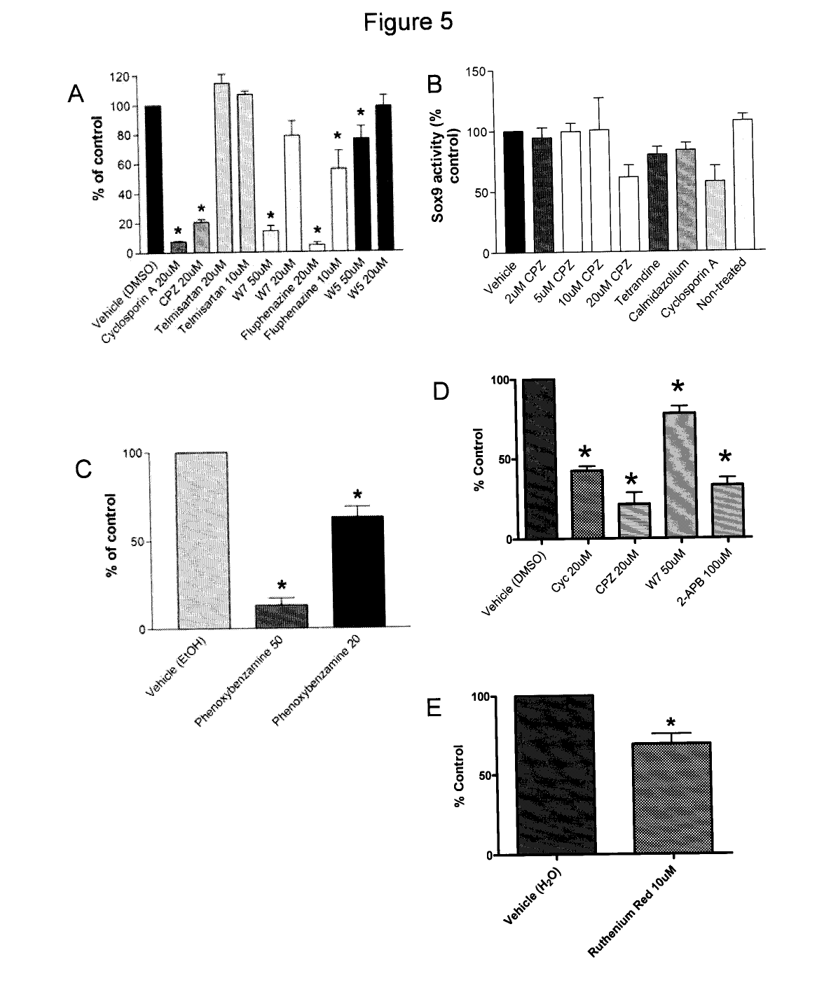

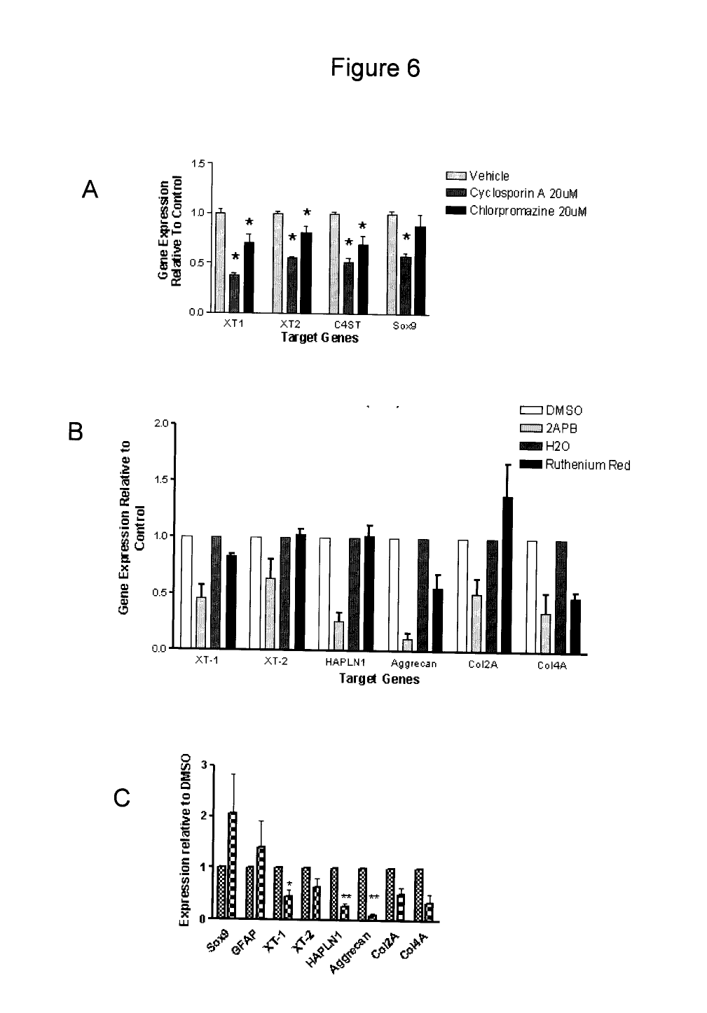

A simple statistical parameter used to validate a screen of this nature is to calculate the Z′ factor that describes the available signal window for an assay in terms of the total separation between the negative and positive controls minus the error associated with each type of control. A reliable screen is indicated by a Z′ value greater than 0.5. The SOX9 reporter assay has a Z′=0.71. Furthermore, the compounds already demonstrated to reduce the expression of SOX9 target genes in primary astrocytes reliably produce significant reductions in luciferase activity using this reporter system (FIG. 5). Similarly, outside or including the luciferase readout, target genes or proteins may be assessed directly from the cultures themselves in order to derive a more detailed indication of the activity of a candidate inhibitor upon SOX9 (FIG. 6).

### EXAMPLE 2

### Sox9 is Associated with Various CNS Disorders

In order to assess the potential role of SOX9 in human CNS injury and disease, SOX9 expression in human cases of hemorrhagic stroke (n=3), ischemic stroke (n=3), traumatic brain injury (n=2) were surveyed. Briefly, human tissues were formalin fixed and either cryosectioned or paraffin embedded and sectioned for histological testing. Sections were stained with the combinations of commonly commercially available differentially fluorescent antibodies including GFAP, CS56, and Sox9 and counterstained with the fluorescent nuclear dye DAPI. SOX9 expression could clearly be observed in GFAP positive astrocytes and in areas rich in CSPGs in samples from human tissues following Ischemic Stroke, TBI, and Spinal cord injury. Uninjured human CNS samples were void of Sox9, GFAP, and CSPG staining. Similarly immunohistochemistry staining of mouse tissues following MCAO and spinal cord injury demonstrates similar profiles as human tissues.

In addition to the rodent model for SCI (described by Gris et al. 2007 Aug. 15; 55(11):1145-55.), a standard middle cerebral artery occlusion (MCAO) model in mice (described by Belayev et al. Brain Res. 1999 Jul. 3; 833(2):181-90.) was used. The MCAO model is used to emulate ischemic stroke in humans. Briefly, an 11 mm length of monofilament nylon coated with Poly-L-lysine is passed into the common carotid artery of the mouse, through the internal carotid artery, and past the middle cerebral artery (MCA), effectively occluding the MCA. The loop of suture is tightened down and the mouse allowed to recover in a warm cage. Effective cerebral blood flow reduction is confirmed by a laser-Doppler flowmetry probe (reductions of 70% or better indicating successful occlusion). After 30 minutes for a moderate injury or 60 minutes for a severe injury, the mouse is re-anesthetized and the nylon suture removed allowing reperfusion. Immunohistochemistry for SOX9, GFAP and CSPG expression was carried out on sections of mouse brain 7 days and 28 days post-injury. Sections of an uninjured mouse, a mouse 7 days following MCAO or 28 days after MCAO were immunostained for SOX9, GFAP, and/or CS56 and counterstained with DAPI. Double labelling of sections with commonly commercially available anti-GFAP antibodies and anti-SOX9 antibodies demonstrates SOX9 expression in reactive astrocytes. High magnification of cells co-expressing GFAP and SOX9 were revealed. Double labelling of sections with CS56 antibodies, which recognizes several different CSPGs, and anti-SOX9 antibodies demonstrate SOX9 expression in cells surrounding regions immunoreactive for CSPGs. Uninjured mouse tissues were void of staining.

The role of SOX9 in the regulation of target scar genes in rodent models of disease was also assessed directly using two mouse strains. The first strain carries floxed SOX9 (exons 2 and 3 of SOX9 surrounded by loxP sites) alleles (SOX9^{flox/flox} mice). The second mouse strain is a transgenic line that ubiquitously expresses the Cre recombinase fused to the mutated ligand binding domain of the mouse estrogen receptor (ER) under the control of chicken beta actin promoter/enhancer coupled to the CMV immediate early enhancer (CAGGCre-ER™ transgenic mice). The mutated ER ligand binding domain of the fusion protein does not bind endogenous estradiol but is highly sensitive to nanomolar concentrations of tamoxifen. Thus, in CAGGCre-ER™ mice the CreER fusion protein is trapped in the cytoplasm of expressing cells. Tamoxifen administration allows the CreER protein to transport to the nucleus where it excises loxP-flanked regions of DNA. Studies have shown that tamoxifen administration to CAGGCre-ER™ transgenic mice results in Cre-mediated genomic recombination in all organs and brain regions examined. The tamoxifen administration in the SOX9flox/flox;CAGGCre-ER™ will ablate the SOX9 coding region rendering the gene non-functional. The SOX9^{flox/flox} mice were bred with CAGGCre-ER™ transgenic mice to generate mice heterozygous for the floxed SOX9 allele and hemizygous for the Cre-ER transgene, as well as mice bearing SOX9^{flox/flox} that carry the Cre-ER transgene. Using these mice, the molecular, cellular and neurological responses to CNS insult can be observed in the absence of SOX9 expression.

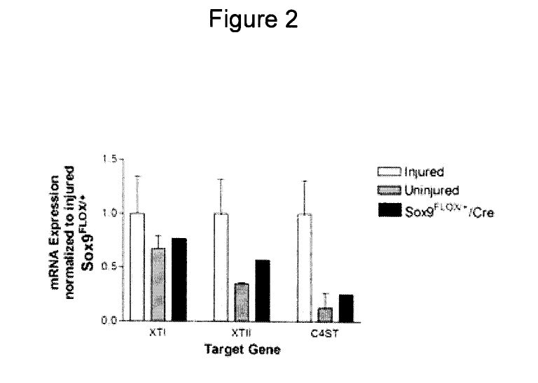

A series of experiments with the SOX9^{flox/+}; Cre-ER™ heterozygous mice were conducted to verify that the Cre transgene is working as expected and to investigate whether knocking out one SOX9 allele has any effects on SOX9 target gene expression. SOX9^{flox/+}; Cre-ER™ and SOX9^{flox/+} mice that do not carry the Cre transgene were treated for one week with a daily dose of tamoxifen (1.5 mg/20 g mouse in corn oil, delivered by gavage). Mice from both groups subsequently underwent an SCI and at one week post-injury they were sacrificed for gene expression studies. The protein isolated from the 5 mm segment of the spinal cord centered on the lesion was analyzed by Western blotting for SOX9 protein content. Specifically, the proteins from uninjured Sox9 heterozygous conditional knockouts, injured ablated SOX9 heterogzygous conditional knockouts or wild type control were analysed by SDS-PAGE, and analyzed by Western blot. The Western Blot was probed with an anti-SOX9 antibody that recognizes the phosphorylated form of the Sox9 and an anti-B-actin antibody as loading control. The heterozygous Sox9 knockout mouse had very reduced expression of Sox9 over the wild-type in injured mice. Further analysis of the RNA derived from the same spinal cord tissue samples by Q-PCR indicates that reduction of SOX9 protein correlates with reduced mRNA levels of SOX9 target genes XT-I, XT-II and C4ST (FIG. 2).

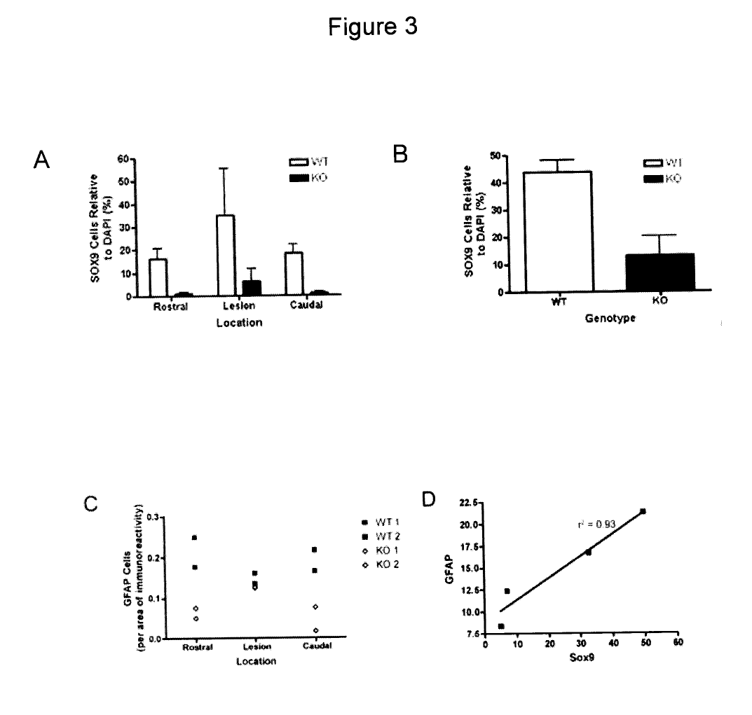

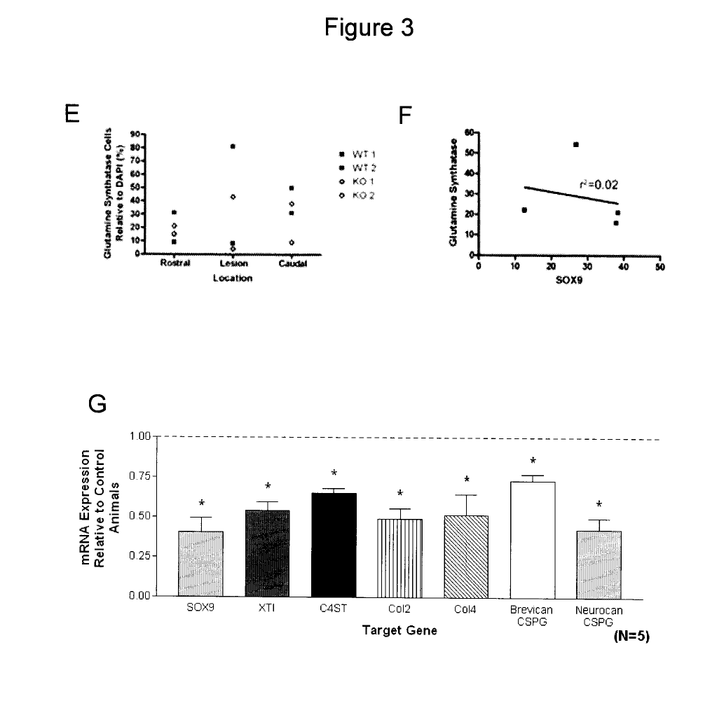

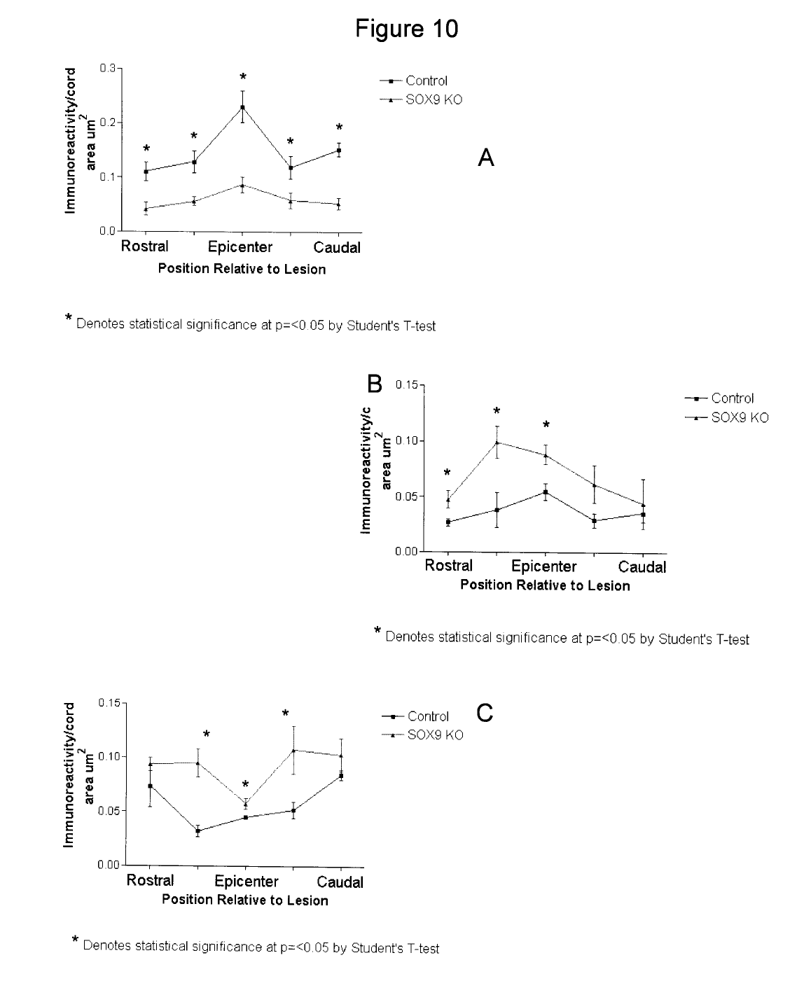

In the case of the homozygous SOX9 conditional knockout mice, knockout of SOX9 following treatment with tamoxifen and spinal cord injury was confirmed with immunohistochemistry and real-time PCR analyses. Longitudinal sections of the spinal cord of wild type and knockout mice were made across the lesion and stained with anti-SOX9 antibodies and counterstained with DAPI. Quantification of the frequency of Sox9 expressing cells indicated a significant decrease in the frequency of SOX9 expressing cells within the lesion in conditional knockout mice versus wild type as well as rostral and caudal to the lesion site (FIG. 3A). With respect to formation of the glial scar, it is the cells immediately rostral and caudal to the lesion that serve the largest role in modulating scar composition. In addition, total frequency of SOX9 positive cells in the spinal cord of the knockout versus wild-type mice indicate an obvious decrease in SOX9 cells (FIG. 3B). It is important to note that due to the fact that the mice are conditional knockouts, all cells of the animal will not necessarily be knocked out, retaining a basal frequency of cells with wild-type characteristics. Further immunohistological analysis was performed to assess SOX9 and GFAP expression as per above. Fluorescent microscopy of sections doublestained for SOX9 and GFAP demonstrated a correlation of SOX9 expression with GFAP. Quantification thereof demonstrates a decreased frequency of GFAP positive cells rostral and caudal to the lesion in knockout mice relative to wild-type controls (FIG. 3C). In addition, linear regression analysis between wild type and SOX9 knockouts demonstrate that there is a strong correlation between the frequency of GFAP expressing cells and SOX9 expressing cells (FIG. 3D). GFAP is recognized as a more specific marker of activated astrocytes, versus more generalized astrocyte markers such as glutamine synthetase. In order to ensure that SOX9 knockout was not impacting frequency of astrocytes, the frequency of glutamine synthetase positive cells was determined from immunohistologically stained sections and demonstrated no obvious differences between wild-type of SOX9 knockouts (FIG. 3E). Linear regression analysis between wild type and SOX9 knockouts did not demonstrate a correlation between the frequency of SOX9 expressing and glutamine synthetase expressing cells (FIG. 3F). FIG. 3G demonstrates further in vivo real-time PCR analysis of the impact of SOX9 knockdown upon scar gene expression. Analysis of SOX9 knockdown and control mice 1 week post spinal cord injury shows that SOX9 knockdown results in a ˜66% reduction in SOX9 mRNA expression compared to control mice. This statistically significant reduction in SOX9 expression (p<0.05 by Student's T-test) is associated with a statistically significant reduction in Xylosyltransferase-I, Chondroitin-4-sulfotransferase, Collagen's 2 and 4, Brevican CSPG and Neurocan CSPG (p<0.05 by Student's T-test) in comparison to control mice. Further immunohistological analysis of the scar lesion demonstrates a significant decrease in CSPG protein (FIG. 10A) within the conditional SOX9 knockout following spinal cord injury, as well as an increase in pro-regenerative laminin protein (FIG. 10B). Finally, there is a significant increase in the protein levels of neurofilament (FIG. 10C) within and adjacent to the lesion indicating a greater presence of nerves and evidence of nerve regeneration.

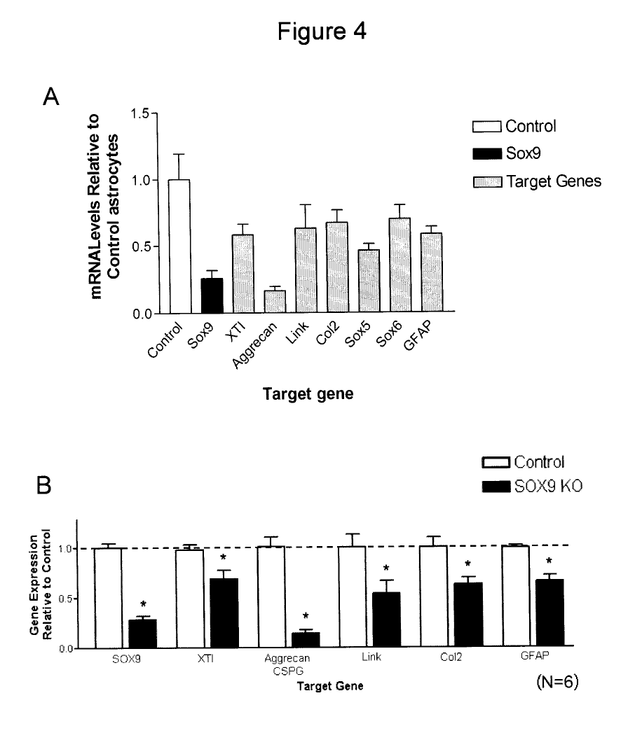

To confirm and validate the relevance of the in vivo evidence with that generated under in vitro conditions using aged astrocyte cultures, astrocytes were cultured from P0 mice that are homozygous for the Floxed SOX9 allele and heterozygous for Cre. Controls are astrocytes from littermates that do not carry Cre. After one week of culture the astrocytes were treated for one week with 1 uM 4-OH-Tamoxifen. A week free of tamoxifen was then allowed to “wash out” the Tamoxifen. The Tamoxifen should cause SOX9 loss only in the cultures that came from the Cre hets. One week after the Tamoxifen treatment was stopped RNA from each culture was collected. The expression of XT-1, Aggrecan, Collagen 2A, Link Protein and GFAP in the tamoxifen treated cultured were reduced to approximately 60% of wildtype levels (FIG. 4A). Link protein hinges the CSPG to the Hyaluronic acid matrix in the ECM. Reduction in GFAP is consistent with a reduction in the state of astrocyte activation. XT-2 and C4ST did not show great reductions. Further, FIG. 4B demonstrates in detail the impact of SOX9 activity reduction upon the of scar gene expression in vitro in SOX9 knockdown and control primary mouse astrocyte cultures using Real Time PCR mRNA analyses. SOX9 knockdown results in a ˜75% reduction in SOX9 mRNA expression compared to control mouse astrocyte cultures. This statistically significant reduction in SOX9 expression (p<0.05 by Student's T-test) is associated with a statistically significant reduction in Xylosyltransferase-I, Aggrecan CSPG, Link protein, Collagen 2, and Glial fibrillary acidic protein expression (p<0.05 by Student's T-test) in comparison to control mouse astrocyte cultures.

To validate that modulation of SOX9 and therefore scar target genes can result in functional behavioral improvements in rodents, behavioral testing was performed on the SOX9 knockout mice treated as above and subject to spinal cord injury as described in Example 8. Briefly, following provision of Tamoxifen or sham through food to homozygous conditional SOX9 knockout mice for one week, mice were given a 70 kdyne contusion injury at T8. After 24 hours (time 0) injured rodents were given a locomotor evaluation as described in Example 8. Any mouse with a score greater than 0.25 was disregarded as the paralysis was incomplete. Once a week they are evaluated by the Basso Mouse scale of locomotor recovery (BMS) which looks at how the ankle joint is moving and other indicies of recovery i.e. a score of 2 indicates plantar placement and is a significant bar to reach.

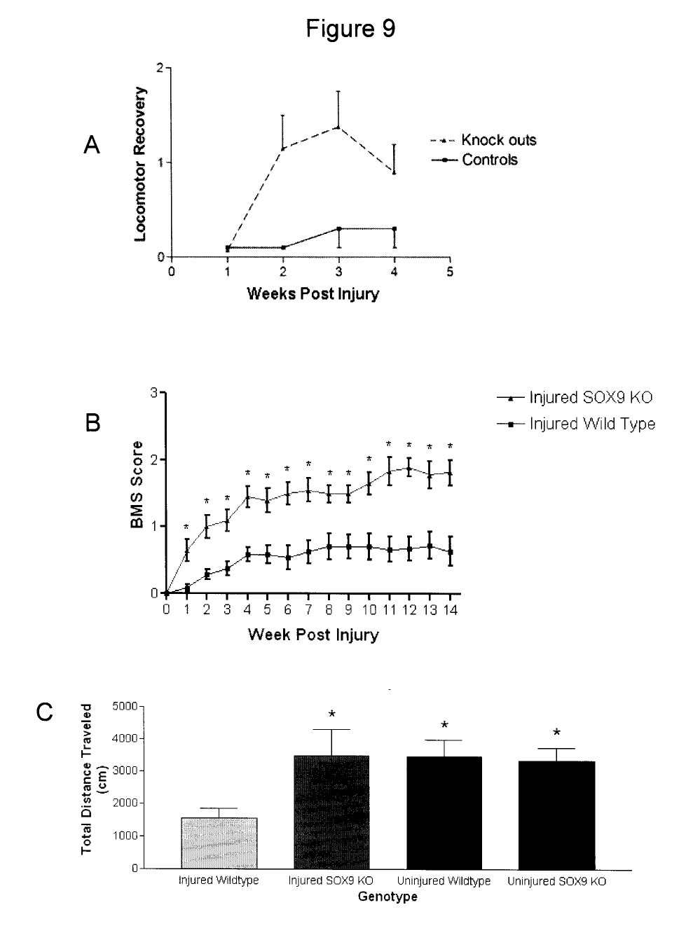

FIG. 9A demonstrates the trend in the behavioral improvement in the mice up to 4 weeks following injury. Clearly, there is improvement in the Sox9 knockout mice relative to control animals indicating that reduction in Sox9 expression can lead to improved behavioral recovery of spinal cord injured mice. Similarly, FIG. 9B demonstrates longer term BMS studies that show a clear improvement over time in the conditional SOX9 knockout mouse following spinal cord injury as well as a continuous trend of improvement over injured littermate controls that plateau in their improvement at approximately 4 weeks post injury. In another test of behavioral recovery, measured as total distance traveled within a rodent activity box, FIG. 9C demonstrates that SOX9 conditional knockout mice display increased locomotion in comparison to control mice. Over 2 hours in a rodent activity box SOX9 conditional knockout mice display increased locomotion in comparison to control mice as determined by 1-way ANOVA (p<0.05). SOX9 conditional knockout mice display the same degree of locomotion as uninjured wildtype control mice and uninjured SOX9 knockout mice as determined by 1-way ANOVA (p<0.05) N=10.

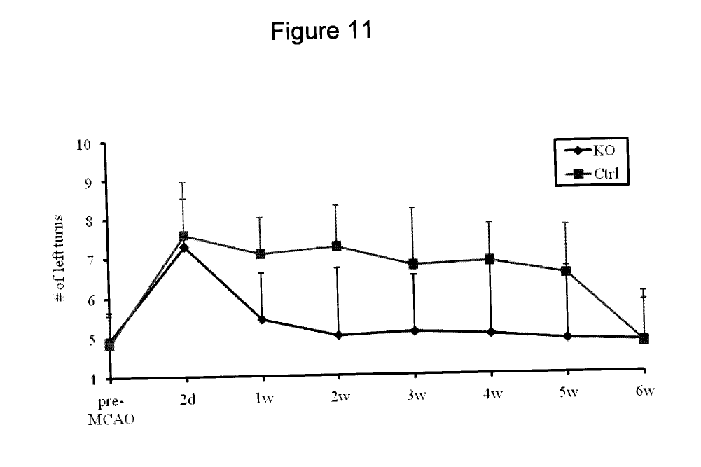

In a further nonlimiting example of SOX9 modulation serving as a general strategy to promote functional CNS regeneration, FIG. 11 demonstrates behavioral recovery of rodents following MCAO (as described further in Example 8), and conditional SOX9 knockdown, as a stroke model of disease. Following MCAO, the histology demonstrates obvious GFAP staining astrocyte cells ipsilateral to injury in the littermate controls indicating activated fibroblasts in sharp contrast to the conditional SOX9 knockout mouse. Further histology and staining for CSPG confirm a decrease in CSPG containing scar within the SOX9 knockout. Subsequent assessment of the number of left turns (out of 10 total turns) overtime during the corner test in knockout (n=14) and control mice (n=12) post MCAO±SD demonstrates a statistically significant difference p<0.05, Newman-Keuls Test over weeks 1 to 5 demonstrating improvements in recovery with the SOX9 knockout (FIG. 11B). Within the corner test behavioural assessment, an uninjured mouse will equally favour its left or right side when confronted with a corner, wherein MCAO rodents with the injury as presented will favour left turns. Similar results were obtained with other behavioral tests including grip strength score and cyclinder test asymmetry.

Taken together with the evidence of the impact of the conditional knockout of SOX9 on the histology of the scar as shown in FIG. 3, and the direct impact upon limiting scar limiting CSPGs and augementing pro-innervation laminin proteins and nerve presence (FIG. 10), locomoter recovery appears to be the result of augmented nerve regeneration, through modulation of scar composition.

### EXAMPLE 3

### Calmodulin Antagonists Impact SOX9 Expression in Astrocytes and SCI

The utility of calmodulin antagonists (inhibitor) to decrease SOX9 target gene expression was evaluated in cultured rat astrocytes transfected with pGL4.1 4×48 Col2a1 prepared as described in Example 1. This plasmid contains 4-48 bp SOX9-binding sites from the Col2A1 enhancer which promote luciferase reporter gene expression in cells where SOX9 is active i.e. in the nucleus. The day after transfection, cells were treated for 24 h with inhibitor in concentrations as described below and in FIG. 5 before the luciferase assay was performed. In the context of Chlorpromazine, there is an apparent dose dependent response to limiting luciferase expression in aged astrocyte cultures optimized clearly at 20 uM (FIG. 5, A,B,D). In addition, the calmodulin inhibitors W7 and W5 also demonstrated does dependent decreases in reporter activity that appeared maximally at 50 uM (FIG. 5A, D). Similarly, Fluphenazine bears structural similarities to W7 and W5 and also demonstrates a dose dependent decrease in reporter activity (FIG. 5A). Similarly, Calmidazolium reduces Sox9 activity as indicated by luciferase expression by approximately 20% (FIG. 5B), while phenoxybenzamine (C) reduces Sox9 activity by up to about 80% and ruthenium red by about 25%.

To explore the impact of these compounds on SOX9 target genes, primary astrocytes from postnatal day I rats were cultured for 2-3 weeks before being treated with either vehicle or a selected antagonist at 20 uM. This time point is the duration within which the astrocytes in culture retain the greatest similarity to stabilized astrocytes of the CNS. After 48 hours of treatment, the cells were harvested and the expression levels of XT-I, XT-II and C4ST were measured by Q-PCR. Treatment of aged primary rat astrocyte cultures with chlorpromazine at 20 uM significantly reduced the expression of XT1, XT2, and C4ST (FIG. 6A). Additionally, Western Blotting of protein extract from the cultures probed with anti-collagen IV demonstrates a reduced level of collagen following treatment with 20 uM chlorpromazine. Collagen IV is a significant scarring extracellular matrix produced by astrocytes and contributing to the inhibitory properties of the glial scar. Studies of longer treatments (1 week) with chlorpromazine produced even more profound reductions in SOX9 target genes without any effects on cell survival.

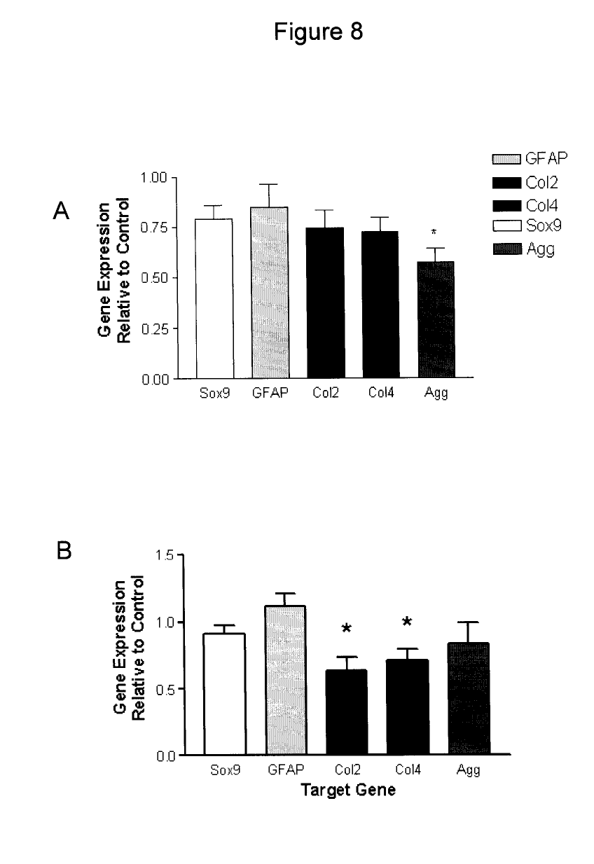

To validate the impact of the compounds on relevant conditions in rodents, a short-term experiment was performed to see whether chlorpromazine might reduce SOX9 target gene expression in vivo. Rats (n=4 per group) underwent a SCI and 2 hours later received either 1 ml of saline ip, or 1 ml of 0.5 mg/ml chlorpromazine ip (2 mg/kg dose) or 1 ml of 5 mg/ml sc cyclosporine A (20 mg/kg dose). Twelve hours later the rats were sacrificed and RNA was isolated from a 5 mm segment of their injured cords centered on the lesion. Analysis of SOX9 target gene expression following spinal cord injury in rats and treatment with either chlorpromazine or control demonstrates a decreased expression of scar generating factors (FIG. 8A). Specifically, the decrease in the expression of GFAP indicates a lower level of astrocyte activation. This data correlates with the evidence produced with the Cre-inducible knock out mouse data in which astrocyte activation was reduced and the number of astrocytes unchanged. In addition, Type II and Type IV collagen expression was reduced and there was a significant decrease in the CSPG Aggrecan over the other markers. Taken together, this indicates that calmodulin antagonist treatment reduces damaging scar gene expression, and in conjunction with the knockout data, suggests that calmodulin antagonists impact is upon SOX9 activity and via calmodulin inhibition.

### EXAMPLE 4

### Peptides Affecting Immunomodulatory Pathways, Calmodulin and Sox9 Transportation

The effect of cyclosporine on SOX9 function was evaluated in cultured rat astrocytes transfected with pGL4.1 4×48 Col2a1. The day after transfection, cells were treated for 24 h with cyclosporine (20 μM) before the luciferase assay was performed. In the context of Cyclosporin A, the application of 20 uM of compounds significantly and dramatically reduces the expression of luciferase in the reporter assay relative to control values (FIG. 5A, B, D). Thus, cyclosporine directly inhibits calmodulin-SOX9 interaction and SOX9 nuclear transport.

In order to confirm that inhibition of SOX9 activity has an effect on glial scar gene expression, stabilized primary astrocytes were treated with vehicle or 20 μM cyclosporine A. After 48 hours of treatment, the cells were harvested and their expression levels of XT-I, XT-II and C4ST measured by Q-PCR. Treatment of aged primary rat astrocyte cultures with Cyclosporin A at 20 uM significantly reduced the expression of XT1, XT2, and C4ST, in addition to Sox9 (FIG. 6A). Additionally, Western Blotting of protein extract from the cultures probed with anti-collagen IV demonstrates a reduced level of collagen following treatment with 20 uM Cyclosporin A. Collagen IV is a significant scarring extracellular matrix produced by astrocytes and contributing to the inhibitory properties of the glial scar. Studies of longer treatments (1 week) with cyclosporine produce even more profound reductions in SOX9 target genes without any effects on cell survival.

To determine the utility of cyclosporine on astrocyte activity in rodent models of disease, short-term experiments were performed to see whether or not cyclosporin A reduces SOX9 target gene expression in vivo. Rats (n=4 per group) underwent a SCI and 2 hours later received either 1 ml of saline ip, 1 ml of 5 mg/ml sc cyclosporine A (20 mg/kg dose). Twelve hours later the rats were sacrificed and RNA was isolated from a 5 mm segment of their injured cords centered on the lesion. Analysis of SOX9 target gene expression following spinal cord injury in rats and treatment with either Cyclosporine A or sham demonstrates a decreased expression of scar generating factors (FIG. 8B). Specifically, Type II and Type IV collagen expression was significantly reduced (˜40% reduction) in addition to the apparent 40% reduction in the CSPG Aggrecan. Taken together, this suggests that cyclosporine A treatment reduces damaging scar gene expression, via a combination of modulating the immune response and calmodulin inhibition.

### EXAMPLE 5

### Peptides Affecting Calmodulin and Sox9 Transportation

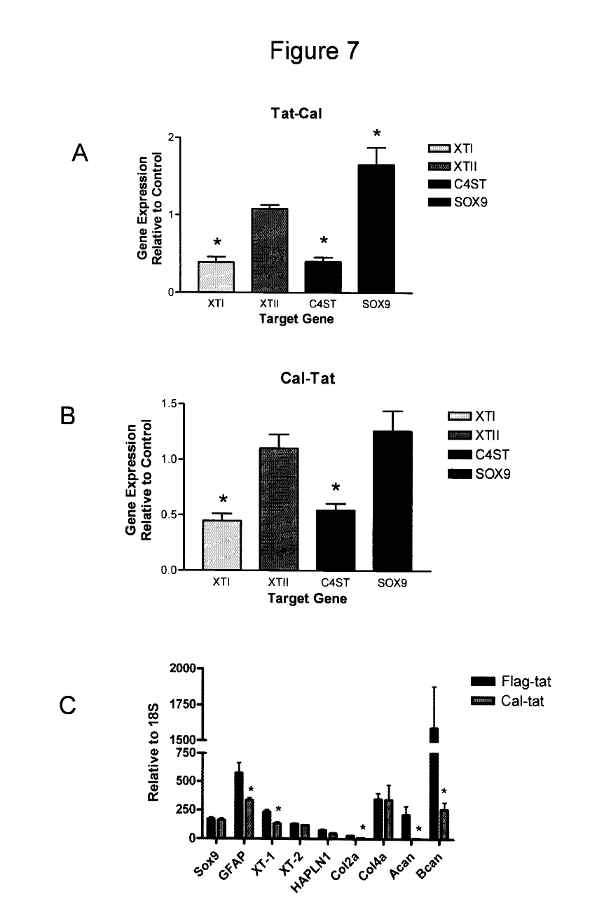

Another approach to modulating the capacity of calmodulin to transport SOX9 into the nucleus is via competitive inhibition of calmodulin binding. To that effect, a novel peptide (SOX-CAL) was generated to specifically block the calmodulin SOX9 binding site. The SOX-CAL peptide sequence, RRPMNAFMVWAQAARRK (SEQ ID NO.8), corresponds with the calmodulin binding sequence in SOX9. To ensure the peptide's entry into the cell, it was synthesized fused to the protein transduction domain of the HIV-1 Tat protein. Primary astrocytes from postnatal day 1 rats were cultured for 2-3 weeks before being treated with 10 μM SOX-CAL peptide. After 48 hours of treatment the cells were harvested and their expression levels of XT-I and C4ST measured by Q-PCR. Treatment of aged primary rat astrocyte cultures with either 10 uM of Cal-TAT or TAT-Cal significantly reduced the expression of XT1, and C4ST as determined by real-time q-PCR (FIG. 7A,B). interestingly, while it appears that TAT-Cal increased the expression of Sox9 in the aged cultures, the SOX9 protein did not appear to be impacting the transcription of the SOX9 target genes suggesting that the SOX9 protein was not making its way to the nucleus as expected. Further assessment of the impact of Cal-TAT upon target gene expression in primary astrocyte cultures demonstrated a significant reduction in GFAP, XT-1, Collagen 2A, and the CSPG core proteins aggrecan and brevican (FIG. 7C). This analysis was performed in contrast to the control FLAG-TAT peptide.

Additionally, Western Blotting of protein extract from the Cal-TAT treated and control cultures probed with anti-collagen IV demonstrates a reduced level of type IV collagen. Collagen IV is a significant scarring extracellular matrix produced by astrocytes and contributing to the inhibitory properties of the glial scar. The fusion of TAT is not expected to have an impact on cell survival and target genes relating to nerve regeneration. The reduced levels of target genes were equivalent regardless of the location of the Tat sequence on either the N- or C-terminal position (FIG. 7) suggesting that the impact of SOX-CAL upon target genes and activity is independent of the configuration of the SOX-CAL peptide sequence and TAT fusion. Additionally, further studies suggest that longer treatments (1 week) with SOX-CAL peptide produce even more profound reductions in SOX9 target genes without any effects on cell survival.

With respect to in vivo applications, as a peptide is likely to have a very short half-life in blood, it may be delivered intrathecally using a miniosmotic pump. The 1002 Alzet osmotic mini pump can deliver drug at a rate of 0.25 μL per hour for two weeks. Pilot experiments using the control peptide FLAG-Tat (this peptide is identical to the SOX-CAL peptide except the amino acid sequence that binds calmodulin has been replaced by the FLAG sequence DYKDDDDK (SEQ ID NO:12) for which commercial antibodies are available) can be performed to estimate the volume of the cord occupied by the infused peptide after 4 hours of drug delivery. This volume estimate will be used to calculate the expected dilution factor of the SOX-CAL peptide in the injured cord and will allow estimation of the concentration of peptide that should be used in the pump.

### EXAMPLE 6

### TRPV Antagonists to Modulate Astrocyte Activity and Scar Composition

In this example, the potential of compounds that modulate calcium channels to modulate SOX9 activity is tested. The compounds were evaluated in cultured rat astrocytes transfected with pGL4.1 4×48 Col2a1. The day after transfection, cells were treated for 24 h with selected inhibitors before the luciferase assay was performed. Phenoxybenzamine demonstrates a dose dependent inhibition of reporter activity at 20 and 50 uM (FIG. 5C). Additionally, Tetrandine reduces luciferase activity by approximately 20% (FIG. 5B). Finally, both Ruthenium Red and 2-APB, the TRPV4 specific channel inhibitors, significantly reduce luciferase production (FIG. 5D,E). It is believed that these compounds inhibited calcium influx, and therefore calmodulin capacity to transport SOX9 to the nucleus and activate the luciferase reporter.

Antagonists to TRPV4 cation channel were tested to determine their ability to decrease the activity of SOX9 in astrocytes. The effect of TRPV4 antagonists on SOX9 function was evaluated in cultured rat astrocytes transfected with pGL4.1 4×48 Col2a1. The day after transfection, cells were treated for 24 h with 2-APB (100 μM) or ruthenium red (10 μM) before the luciferase assay was performed. The broad-spectrum transient receptor potential (TRP) channel antagonist 2-APB inhibited SOX9 activity by ˜70%, while the vanniloid subfamily-specific TRP antagonist ruthenium red inhibited SOX9 activity by ˜25%. With respect to SOX9 target genes, Q-PCR analysis of aged primary astrocyte cultures was performed to determine the impact of the TRPV4 antagonists on SOX9. Treatment with ruthenium red specifically reduces aggrecan and Col4a expression by 40% and 50%, respectively (FIG. 6B), while 2-APB reduces XT-1, HAPLN1, aggrecan, Col2a, and Col4a by 50%, 70%, 80%, 50%, and 60% respectively (FIG. 6B, C), in rat primary astrocytes after a 48 hour exposure (n=3). Collagen IV is a significant scarring extracellular matrix produced by astrocytes and contributing to the inhibitory properties of the glial scar. Based on the role that the TRP channels serve in modulating calcium levels, and therefore calmodulin activity, the effect of the TRP channel blockers on SOX9 activity appears to be indirect.

### EXAMPLE 7

### In Vivo Experiments with Chemical Compounds Affecting Calmodulin

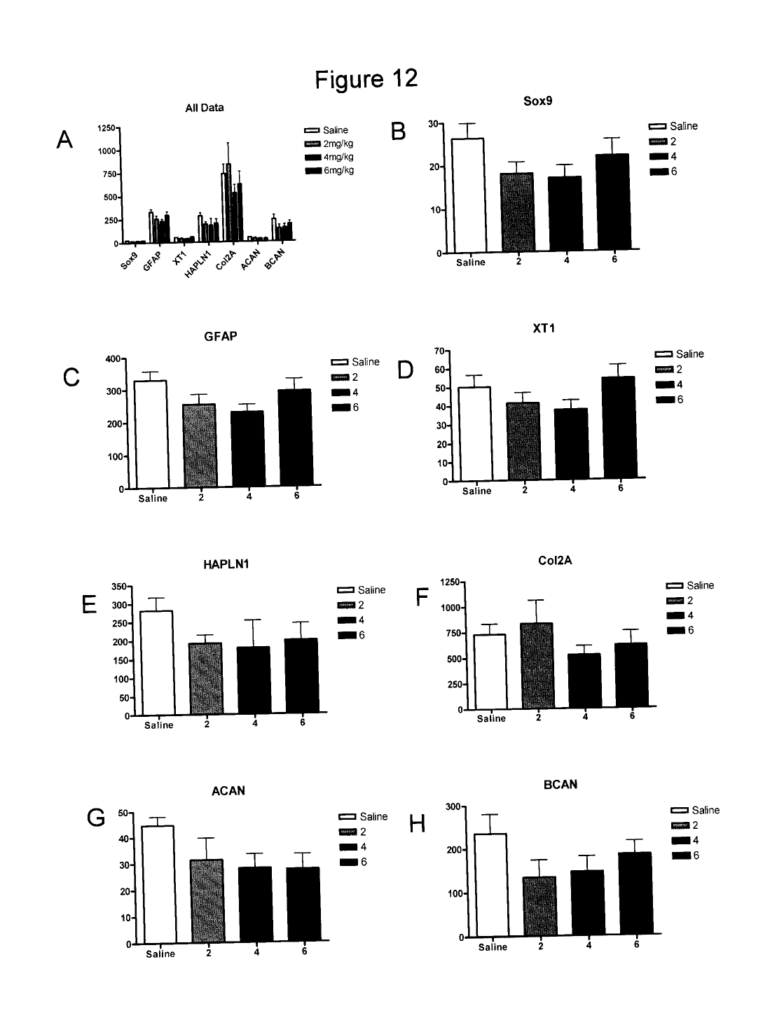

In the context of rodent studies, administration of chlorpromazine was assessed using delivery by intraperitoneal injection (as described in Example 3) and intrathecal miniosmotic pump (as described in Example 5). In summary, three doses of chlorpromazine were given i.p. (2 mg/kg, 4 mg/kg and 6 mg/kg) once a day for 7 days. The rats were then sacrificed and realtime pCR analysis of SOX9, GFAP. XT1, link protein, collagen 2A, aggrecan and brevican was conducted on a spinal cord sample normalizing to 18S. FIG. 12 demonstrates that following spinal cord injury in rats, delivery of chlorporomazine by IP injection reduces the expression of SOX9 target genes (FIG. 12A) including SOX9 (FIG. 12B), GFAP (FIG. 12C), XT-1 (FIG. 12D), HAPLN1 (FIG. 12E), type 2A Collagen (FIG. 12F), Aggrecan (FIG. 12G) and Brevican (FIG. 12H). Similarly, the results demonstrate that there is an apparent impact on target gene expression at 2 and 4 mg/ml administration.

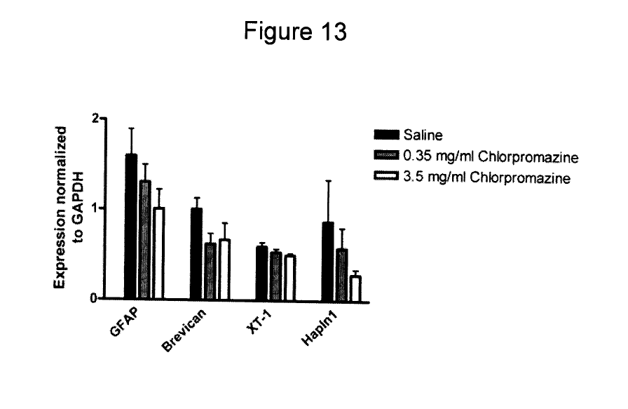

To assess, the intrathecal miniosmotic pump as a means of compound delivery, spinal cord-injured rats received either saline, 0.35 mg/ml or 3.5 mg/ml of chlorpromazine for 7 days. At 7 days the levels of SOX9 target gene expression including GFAP, Brevican, XT-1 and Hapln1 were measured by quantitative RT-PCR as previously described. The evidence presented in FIG. 13 demonstrates the dose response outcome associated with Chlorpromazine and intrathecal minipump delivery and suggests an improved anticipated outcome at 3.5 mg/ml of drug.

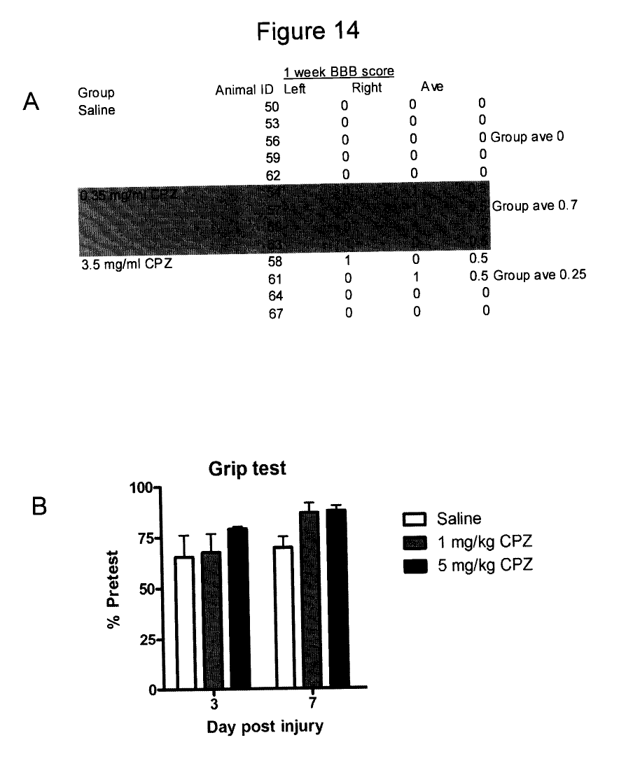

As examples of the general utility of chemical compounds for general CNS damage and repair, chlorpromazine was assessed in rodent models of spinal cord injury and stroke (MCAO). FIG. 14A demonstrates results of behavioral testing using the BBB scale as described in Example 8. Briefly, rats were subject to spinal cord injury as described and administered chlropromzaine at 0.35 mg/ml and 3.5 mg/ml for 7 days via the intrathecal miniosmotic pump. At 7 days, 4 rats were assessed for behavioral recovery, prior to being sacrificed for gene expression analyses as described above. FIG. 14A shows that none of the saline control treated rats demonstatesd any improvement in locomoter function with either their left or fight foot. At both doses of chlorpromazine, non-zero scores were identified indicating evidence of functional recovery as demonstrated by foot implant.

To demonstrate efficacy in stroke models, MCAO was performed on mice as described in Example 8, and doses of 1 mg/kg or 5 mg/kg of chlorpromazine were administered daily by intraperitoneal injection starting at 24 hours post-injury. At 3 days and 7 days post-injury, recovery of the damaged side was assessed by grip testing. FIG. 14B demonstrates that at 5 mg/kg of chlorpromazine, there is evidence of approximately 15% improvement over saline control at 3 days post injury, and further improvement to approximately 20% over saline control by 7 days post injury. This provides evidence that chemical compounds that modulate SOX9 activity can improve behavioral function following general CNS damage including that associated with spinal cord injury and stroke.

### EXAMPLE 8

### Rodent Experiments with Therapeutic Compounds

Further to Example 7, the potency of the SOX9 inhibitors (chlorpromazine, cyclosporin, SOX-CAL, ruthenium red and 2-APB) may be assessed in a rodent model of SCI. Pilot studies are conducted to determine the best dosing of the drug under study. In the context of rodent studies, dosing for 2-APB begins at 2 mg/kg, ip. For Ruthenium Red, the rodent dosing begins at 1 mg/kg, ip. For cyclosporine, rodent dosing begins at 10 mg/kg, sc. Additionally, doses up to 50 mg/kg are common in the literature. For chlorpromazine, rodent dosing begins at 2 mg/kg, ip. Briefly, mice are anesthetized with 1.5% halothane and a laminectomy is performed to expose the 4th thoracic spinal segment. A modified aneurysm clip calibrated to deliver a 3 g force is placed extradurally around the cord and closed for 60 seconds. This model of SCI closely replicates the key pathophysiological features of human injury by producing prolonged, rapidly applied, extradural compression. This model produces mechanical injury and secondary damage by microvasculature disruption, hemorrhage, ischemia, increases in intracellular calcium, calpain activation, progressive axonal injury and glutamate toxicity. Alternately, rats will receive a contusion injury at T10 (10^{th} thoracic level) using the Infinite Horizon impactor. Beginning at 48 hours after injury the animals will be treated with either vehicle (controls) or with a SOX9 inhibitor according to the dosing schedule outlined. For these studies, the drugs will be administered for 2 weeks, a time point at which the scar has been well-developed in the rat. The animals (n=6 per group) will then be sacrificed and processed for RNA analyses. RNA will be isolated from a 5 mm segment of spinal cord centered on the spinal lesion as previously described (Gris 2009). Q-PCR will be carried out on the RNA samples to evaluate the mRNA levels of SOX9 target genes that will include XT-I, XT-II, C4ST, Collagen 2, aggrecan and link protein. Three doses of each drug will be evaluated, increasing in 2-fold increments.

Compounds and concentrations that demonstrate the strongest reductions in SOX9 target gene expression will be tested in a long-term study as follows. In these studies rats (n=16 per group) will receive the SOX9 inhibitor for 6 weeks at which time half of the animals will be processed for SOX9 target gene expression by Q-PCR (as described above) and half will be processed for immunohistochemistry. The immunohistochemistry will allow correlation of changes in SOX9 target gene expression with alterations in the amount of CSPGs, collagen and laminin at the scar. In addition, during the six weeks of treatment the rats will undergo locomotor testing to evaluate any benefits in neurological recovery that might be attributed to the test compounds. Locomotor recovery, chronic pain syndromes and autonomic function will be assessed in treated and control mice. Motor function will be evaluated once per week using the Basso, Beattie and Bresnahan (BBB) scale for scoring hind limb function (Basso 1995). The presence of mechanical allodynia (a pain syndrome in which innocuous stimuli are perceived as painful) will be assessed once a week by stimulating the backs of the mice with a modified 1.569 mN Semmes Weinstein monofilament at, and rostral, to the level of the injury. This will be done in an open cage and the number of avoidance responses to ten stimuli will be tabulated. After a 3 g clip, SCI mice routinely develop avoidance behaviors (attempts to escape, vocalization, jumping, flinching and/or attempting to bite the filament). Finally autonomic function will be assessed by measuring the degree of autonomic dysreflexia in animals before being sacrificed. Autonomic dysreflexia is characterized by episodic hypertension triggered by sensory stimulation below the level of the spinal lesion and is thought to be due to the loss of descending inhibitory inputs and the generation of abnormal reflexes in the injured cord (Brown 2006). The clip SCI in mice reliably produces autonomic dysreflexia as measured by increases in blood pressure in response to colon distension that correlates to the degree of SCI.

The therapeutic impact of the selected compounds on other CNS injuries (MCAO or TBI) will be performed as outlined below. Animals will then be sacrificed at 1, 3, 6 and 8 weeks post-injury for histological (n=4), gene expression (n=4) analyses. Analyses of neural plasticity (n=8) will be performed at 6 weeks post-injury and behavioural analyses ill be assayed at 3, 4, 5 and 6 weeks post-injury.

TBI injury model: These experiments will be done using the fluid percussion injury model of TBI. Fluid percussion injury (FPI) is the most common clinically relevant model of TBI with over a decade of literature supporting its use in rats and mice. In brief, mice will be anesthetized and placed in a sterotactic head holder. After reflecting back the scalp a 2.0 mm diameter right-sided craniectomy will be performed 0.5 mm lateral to the sagittal suture and 0.5 mm caudal to the bregma. A 2.0 mm (inner diameter) injury cap will then be placed over the craniectomy and secured with glue. After a 24 hour period to allow the mice to recover from the surgery they will be re-anesthetized and connected to the FPI device by high-pressure tubing (2.0 mm inner diameter). An injury magnitude of approximately 3.5 atm will be delivered to each mouse. Immediately after injury the animals will be disconnected from the FPI device and allowed to recover on a heating pad. Evidence using antibody-based approaches in rodent models of TBI (Utagawaa, Bramletta, Danielsa, Lotockia, Dekaban, Weaver, Dietrich, 2008, Brain Research, 1207: 155-163) that we have demonstrated impact SOX9 expression lend credence with the support of the proof of concept mechanism data presented herein, and the in vivo evidence with spinal cord injury and MCAO demonstrated herein, that the strategies of SOX9 modulation presented herein are applicable to TBI applications.

Stroke injury model: A standard MCAO mouse model will be utilized. Briefly, an 11 mm length of monofilament nylon coated with Poly-L-lysine is passed into the common carotid artery, through the internal carotid artery, and past the middle cerebral artery (MCA), effectively occluding the MCA. The loop of suture is tightened down and the mouse allowed to recover in a warm cage. Effective cerebral blood flow reduction is confirmed by a laser-Doppler flowmetry probe (reductions of 70% or better indicating successful occlusion). After 30 minutes for a moderate injury or 60 minutes for a severe injury, the mouse is re-anesthetized and the nylon suture removed allowing reperfusion.

Analyses—Histological and Gene Expression: Cresyl violet staining of these sections and ImagePro software will permit determination of the area of infarct or lesion per section and whether treatment results in changes in lesion volumes after SCI, MCAO or TBI. Tissue samples of equivalent size will be “punched out” of the damaged side of the cortex centering on the lesion epicentre using stereotactic or-ordinates as defined by the histological analyses. XT-I, XT-II, C4ST, laminin and fibronectin gene expression will be assessed by Q-PCR and slot blot analysis of the RNA and protein, respectively isolated from these tissue samples. Fibronectin protein will not be measured because hemorrhage into the injury site results in high levels of fibronectin not associated with new gene expression.

Analyses—neural plasticity: As reviewed above, a major mechanism of recovery of neurological function after CNS injury is through increased neural plasticity whereby uninjured neurons form new connections on deafferented neurons and serve functions previously carried out by the injured neurons. For example, in uninjured animals the majority of corticofugal fibers project ipsilaterally in the midbrain. However after MCAO biotinylated dextran amine (BDA) tracing of cortical projections from the uninjured side reveals increased corticolfugal fibers projecting to the contralateral midbrain. Similarly, in the cervical spinal cords of uninjured animals most corticospinal fibers originate from the contralateral cortex. BDA tracing after MCAO reveals an increased number of fibers from the undamaged cortex projecting ipsilaterally in the cervical cord. These results indicate that MCAO is followed by a period of plasticity during which time axons from the undamaged cortex may project into territories normally traversed and innervated by the side of the cortex damaged by MCAO. Neuronal plasticity in treated and untreated (negative control) mice will be assessed by tracing corticofugal axons contralateral to the occluded MCA or to the site of TBI. A burr hole will be made in the skull overlying the sensorimotor cortex in treated and untreated mice 6 weeks after MCAO. BDA will be injected at 7 sites (0.5 μl of 10% BDA in PBS) at a depth of 1.5 mm from the cortical surface. Two weeks after BDA injection mice will undergo cardiac perfusion and coronal and transverse sections made of their brains and cervical spinal cords. After incubation with an avidin-biotin-peroxidase complex the BDA will be visualized by a diaminobenzidine reaction. An increase in BDA-labeled fibers projecting into the contralateral midbrain or ipsilateral cervical spinal cord in treated versus untreated mice will indicate that the treatment increases structural plasticity.

Analyses—Behavioral outcomes of TBI: Three well established behavioral tests will be used to monitor neurological impairment/recovery in uninjured mice and injured mice after TBI with and without prior SOX9 ablation: 1) Deacon/Rawlins paddle pool, 2) Elevated plus maze and 3) Crawley box for social behavior.

Analyses—Behavioral outcomes MCAO: Three well established behavioral tests will be used to monitor neurological impairment/recovery in uninjured mice and injured mice after MCAO with and without prior SOX9 ablation: 1) an adhesive removal test, 2) a pole test and 3) a staircase test. Better performance on these tests by treated mice will indicate that treatment following injury improves neurological recovery in mice.

We claim:

1. A method of treating a pathological condition associated with proteoglycan production or modulation in a mammal comprising administering to the mammal an agent selected from the group of a calmodulin antagonist and a transient receptor potential (TRP) channel inhibitor.

2. The method as defined in claim 1, wherein the pathological condition is a condition involving inhibition of neuronal growth or neuronal plasticity.

3. The method of claim 2, wherein the condition is selected from the group consisting of spinal cord injury, traumatic brain injury, neurodegenerative disease, Friedreich's ataxia, spinocerebellar ataxia, Alzheimer's disease, Parkinson's Disease, Lou Gehrig's Disease (ALS), demyelinative disease, multiple sclerosis, transverse myelitis resulting from spinal cord injury, inflammation, and disease associated with retinal neuronal degeneration.

4. The method of claim 1, wherein the calmodulin antagonist is selected from the group consisting of alpha-adrenergic blockers, phenothiazines, naphthalenesulfonamides, ACE inhibitors, alkaloids and pharmaceutically acceptable salts thereof.

5. The method of claim 1, wherein the calmodulin antagonist is selected from the group consisting of phenoxybenzamine, Prazosin, Terazosin, Doxazosin, Tamsulosin, chlorpromazine, calmidazolium, E6 Berbamine, CGS 9343B, trifluoperazine, fluphenazine, cyclosporine, rapamycin, FK506, A7, J8, W-5, W-7, W-13, Losartan, Valsartan, Irbesartan, Candesartan and Tetrandrine.

6. The method of claim 1, wherein the transient receptor potential (TRP) channel inhibitor is selected from the group consisting of 2-APB, ruthenium red, citral, RN9893, RN1734, and pharmaceutically acceptable derivatives or salts thereof.

7. The method of claim 1, wherein the calmodulin antagonist is a calmodulin-binding peptide.

8. The method of claim 7, wherein the calmodulin-binding peptide is represented by the formula:
X¹RP−spacer−RX¹X²
wherein X¹ is a positively charged amino acid such as arginine (R), lysine (K) or histidine (H); X² is a positively charged amino acid such as arginine (R), lysine (K) or histidine, or is no amino acid; and the spacer comprises from about 8-12 amino acid residues.

9. The method of claim 8, wherein the peptide is selected from the group consisting of
(SEQ ID NO. 1)
KRPMNAFIVWSRDQRRK,
(SEQ ID NO. 2)
KRPMNAFMVWSRGQRRK,
(SEQ ID NO. 3)
KRPMNAFMVWSRAQRRK,
(SEQ ID NO. 4)
KRPMNAFMVWSQIERRK,
(SEQ ID NO. 5)
KRPMNAFMVWSKIERRK,
(SEQ ID NO. 6)
KRPMNAFMVWSQHERRK,
(SEQ ID NO. 7)
KRPMNAFMVWAKDERRK,
(SEQ ID NO. 8)
KRPMNAFMVWAQAARRK,
(SEQ ID NO. 9)
RRPMNAFMVWAKDERKR,
(SEQ ID NO. 10)
KRPMNAFMVWSSAQRR
and
(SEQ ID NO. 11)
KRPMNAFMVWARIHR.

10. The method of claim 9, wherein the peptide is modified to incorporate one or more groups which stabilize, protect or facilitate the delivery of the peptide to a target site.

11. The method of claim 1, wherein the agent is administered to the mammal in combination with a pharmaceutically acceptable carrier.

12. The method of claim 1, wherein the agent is administered in a dosage in the range of about 1 ug-100 mg.

13. The method of claim 1, wherein the agent is administered in combination with one or more additional agent that modulate SOX9 expression or activity.

14. A calmodulin-binding peptide represented by the formula:
X¹RP−spacer−RX¹X²
wherein X¹ is a positively charged amino acid such as arginine (R), lysine (K) or histidine (H); X² is a positively charged amino acid such as arginine (R), lysine (K) or histidine, or is no amino acid; and the spacer comprises from about 8-12 amino acid residues.

15. The peptide of claim 14, selected from the group consisting of:
(SEQ ID NO. 1)
KRPMNAFIVWSRDQRRK,
(SEQ ID NO. 2)
KRPMNAFMVWSRGQRRK,
(SEQ ID NO. 3)
KRPMNAFMVWSRAQRRK,
(SEQ ID NO. 4)
KRPMNAFMVWSQIERRK,
(SEQ ID NO. 5)
KRPMNAFMVWSKIERRK,
(SEQ ID NO. 6)
KRPMNAFMVWSQHERRK,
(SEQ ID NO. 7)
KRPMNAFMVWAKDERRK,
(SEQ ID NO. 8)
KRPMNAFMVWAQAARRK,
(SEQ ID NO. 9)
RRPMNAFMVWAKDERKR,
(SEQ ID NO. 10)
KRPMNAFMVWSSAQRR
and
(SEQ ID NO. 11)
KRPMNAFMVWARIHR.

16. The peptide of claim 14, modified to incorporate one or more groups which stabilize, protect or facilitate the delivery of the peptide to a target site.

17. The peptide of claim 16, modified to incorporate a protecting group at an internal site or at a terminal end thereof.

18. The peptide of claim 16, modified to include a peptide that facilitates delivery to a target site selected from the group consisting of a TAT peptide, an MPG peptide, Wr-T and Pep-1 peptide.

19. A composition comprising a peptide as defined in claim 14.

20. An article of manufacture comprising packaging and a composition comprising at least one of a calmodulin antagonist, a calmodulin-binding protein and a transient receptor potential (TRP) channel inhibitor, wherein the packaging is labeled to indicate that the composition is for the treatment of a condition associated with proteoglycan production or modulation.
